(19) United States
(12) Patent Application Publication
(10) Pub. No.: US 2012/0035596 A1
(43) Pub. Date: Feb. 9, 2012

(54) Disposable Drive Interface for Longitudinal Movement of an Elongate Medical Device

(76) Inventor: Troy T. Tegg, Elk River, MN (US)

(21) Appl. No.: 12/850,418

(22) Filed: Aug. 4, 2010

(51) Int. Cl. A61B 17/00

(52) U.S. Cl. 606/1

(57) ABSTRACT

A disposable drive interface kit is provided for controlling longitudinal movement of an elongate medical device such as a catheter. The kit includes a detachable housing configured for removable attachment to a drive housing from which a driving member projects. The detachable housing has inner side that faces the drive housing upon attachment to the drive housing and an outer side defining a channel having a bottom and first and second opposed sides extending from the bottom and configured to receive the elongate medical device. A driven member configured for movement by the driving member and an idler member extend into the channel on the first and second sides, respectively, and define first and second opposed surfaces configured to engage the elongate medical device.

### BACKGROUND OF THE INVENTION

a. Field of the Invention

This invention relates to a system for controlling longitudinal movement of elongate medical devices such as catheters. In particular, the instant invention relates to a disposable drive interface kit having relatively few parts that enables transfer of motive force from a drive system to the medical device while permitting reuse of the drive system.

b. Background Art

Catheters are used for to perform a variety of tasks within human bodies and other bodies including the delivery of medicine and fluids, the removal of bodily fluids and the transport of surgical tools and instruments. In the diagnosis and treatment of atrial fibrillation, for example, catheters may be used to deliver electrodes to the heart for electrophysiological mapping of the surface of the heart and to deliver ablative energy to the surface among other tasks. Catheters are typically routed to a region of interest through the body's vascular system. In a conventional approach, an introducer is used to puncture the skin surface and a sheath having an inner diameter greater than the outer diameter of the catheter is threaded through the vasculature to a region of interest. The catheter is then moved longitudinally through the sheath to the region of interest. The catheter may be advanced and retracted manually by the clinician. Alternatively, or in addition, the catheter may be advanced and retracted using electromechanical drive systems.

Several conventional drive systems are shown in U.S. Pat. No. 7,276,044. In one embodiment of a drive system (shown in FIG. 14 of U.S. Pat. No. 7,276,044), a catheter is received within a channel defined in the upper surface of a housing for the drive system and is placed between opposed wheels with one of the wheels driven by a motor. The disclosed embodiment has the drawback that all or substantially all of the drive system is generally disposed of after use. Because much of the drive system—including the housing—comes into contact with the catheter, the drive system must be disposed of after use or sterilized using conventional sterilization techniques such as ethylene oxide (EtO) gas or irradiation. Because the drive system generally includes sensitive electronic components, sterilization is difficult, expensive and often not cost effective.

The inventor herein has recognized a need for a disposable drive interface kit for longitudinal movement of an elongate medical device such as a catheter that will minimize and/or eliminate one or more of the above-identified deficiencies.

### BRIEF SUMMARY OF THE INVENTION

It is desirable to provide a disposable drive interface kit for controlling longitudinal movement of an elongate medical device such as a catheter. In particular, it is desirable to provide a disposable drive interface kit having relatively few parts that enables transfer of motive force from a drive system to the medical device while permitting reuse of the drive system.

In accordance with one embodiment of the invention, a disposable drive interface kit for controlling longitudinal movement of an elongate medical device is configured for attachment to a drive housing from which a driving member projects. The kit includes a detachable housing configured for removable attachment to the drive housing. The detachable housing has an inner side and an outer side. The inner side is configured to face the drive housing upon attachment of the detachable housing to the drive housing. The outer side defines a channel having a bottom and first and second opposed sides extending from the bottom and configured to receive the elongate medical device. The kit further includes a driven member configured for movement by the driving member and extending into the channel on the first side of the channel. The driven member defines a first surface configured to engage the elongate medical device. The kit further includes an idler member extending into the channel on the second side of the channel. The idler member defines a second surface opposing the first surface and configured to engage the elongate medical device. In accordance with several embodiments of the invention, the kit may further include a coupling member configured for attachment to one of the detachable housing and the drive housing and configured to support a tubular body such as an introducer sheath through which the elongate medical device may be moved longitudinally.

In accordance with another embodiment of the invention, a method of controlling longitudinal movement of an elongate medical device is provided. The method includes the step of attaching a detachable housing to a drive housing from which a driving member projects. The detachable housing has an inner side and an outer side. The inner side is configured to face the drive housing upon attachment of the detachable housing to the drive housing and the outer side defines a channel having a bottom and first and second opposed sides extending from the bottom and configured to receive the elongate medical device. The method further includes the step of engaging a driven member with the driving member for rotation therewith. The driven member extends into the channel on the first side of the channel and opposes an idler member extending into the channel on the second side of the channel. The method further includes the steps of inserting the elongate medical device into the channel between opposed surfaces of the driven member and the idler member and actuating the drive member to move the elongate medical device in a longitudinal direction. Finally, the method includes the steps of removing the elongate medical device from the channel and detaching the detachable housing from the drive housing.

A drive interface kit in accordance with the present invention is advantageous because it has relatively few components and allows reuse of various drive system components. The drive interface kit provides a mechanism for physically separating those components that come into contact with the catheter or similar medical device from other drive system components. In this manner, the other drive system components and be reused with a resulting savings in terms of costs and resources.

The foregoing and other aspects, features, details, utilities and advantages of the present invention will be apparent from reading the following description and claims, and from reviewing the accompanying drawings.

### BRIEF DESCRIPTION OF THE DRAWINGS

FIG. 1 is a perspective view of a drive system including a drive interface kit in accordance with one embodiment of the present invention.

FIG. 2 is an exploded perspective view of the drive system of FIG. 1.

FIG. 3 is a plan view of a portion of the drive interface kit of FIG. 1.

FIG. 4 a perspective view of a drive system including a drive interface kit in accordance with another embodiment of the present invention.

FIG. 5 is a flow chart illustrating a method for controlling longitudinal movement of an elongate medical device in accordance with one embodiment of the present invention.

### DETAILED DESCRIPTION OF EMBODIMENTS OF THE INVENTION

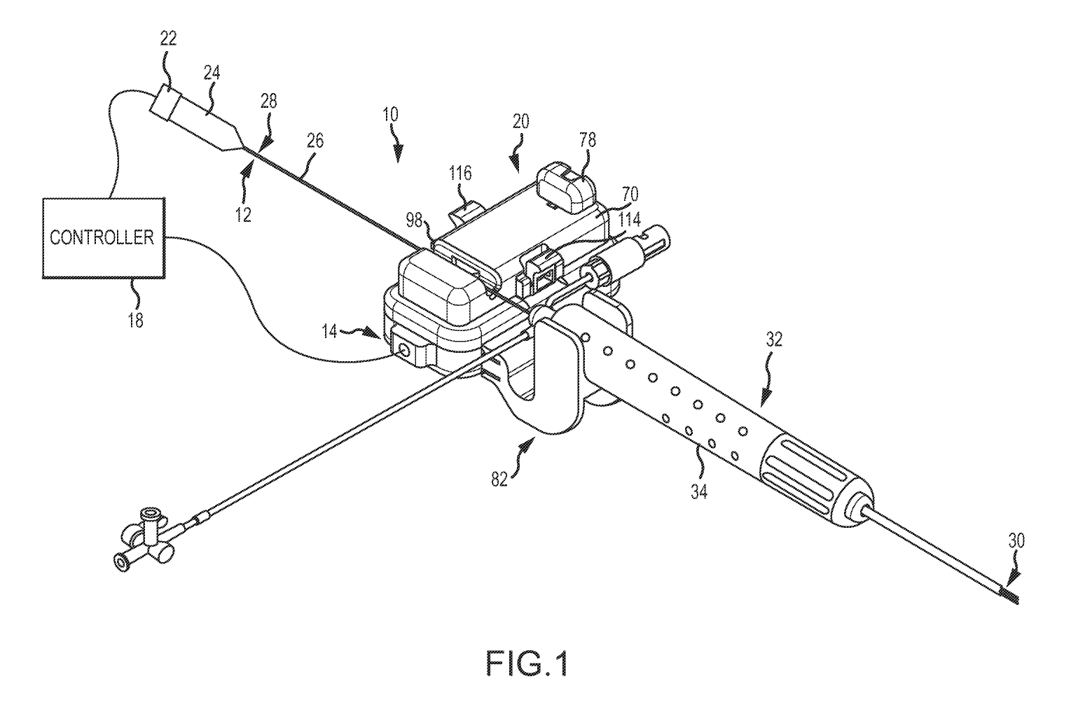

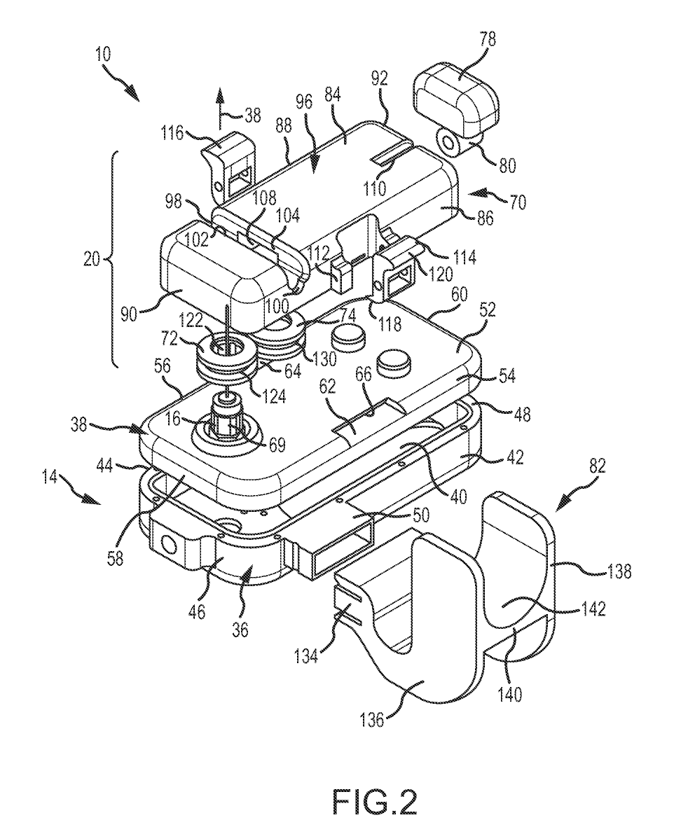

Referring now to the drawings wherein like reference numerals are used to identify identical components in the various views, FIG. 1 illustrates a drive system 10 for controlling longitudinal motion of an elongate medical device 12. Drive system 10 may include a drive housing 14 from which a driving member 16 projects (best shown in FIG. 2), a controller 18 and a drive interface kit 20 in accordance with one embodiment of the invention.

Device 12 is provided for use in diagnostic or treatment procedures on a body. Device 12 may comprise a catheter for delivery of medicine or fluids to a region of interest in a body, removal of bodily fluids and/or transporting surgical tools or instruments within the body. Device 12 may comprise, for example, an electrophysiological (EP) catheter for use in gathering EP data associated with cardiac tissue. Device 12 may alternatively comprise an intracardiac electrocardiography (ICE) catheter for generating an internal image of the heart such as one of the catheters sold by St. Jude Medical, Atrial Fibrillation Division, Inc. under the registered trademark “VIEWFLEX.” Device 12 may alternatively comprise an ablation catheter for providing ablation energy (e.g., radiofrequency, ultrasound, cryogenic, laser or other light) to tissue within the body. Device 12 may include a cable connector or interface 22, a handle 24, and a shaft 26 having a proximal end 28 and a distal 30 end (as used herein, “proximal” refers to a direction toward the end of the catheter near the clinician, and “distal” refers to a direction away from the clinician and (generally) inside the body of a patient). Device 12 may also include other conventional components not illustrated herein and particular to the application of device 12 including, for example, electrodes, sensors and corresponding conductors or leads.

Connector 22 provides mechanical, fluid and electrical connection(s) for device 12. Connector 22 may provide connections to, for example, an irrigation fluid pump or an ablation generator. Connector 22 is conventional in the art and may be disposed at a proximal end of device 12.

Handle 24 provides a location for the clinician to hold device 12 and may further provides means for steering or guiding shaft 26 within the body. For example, handle 24 may include means to change the length of a guidewire extending through device 12 to distal end 30 of shaft 26 to steer shaft 26. Handle 24 is also conventional in the art and it will be understood that the construction of handle 24 may vary.

Shaft 26 is an elongated, tubular, flexible member configured for movement within the body. Shaft 26 may support electrodes, sensors, associated conductors, and possibly additional electronics used for signal processing or conditioning. Shaft 26 may also permit transport, delivery and/or removal of fluids (including irrigation fluids and bodily fluids), medicines, and/or surgical tools or instruments. Shaft 26 may be made from conventional materials such as polyurethane and defines one or more lumens configured to house and/or transport electrical conductors, fluids or surgical tools. Shaft 26 may be introduced into a blood vessel or other structure within the body through a conventional introducer sheath 32 or another tubular body through which device 12 may be moved longitudinally. Shaft 26 may then be steered or guided through the body to a desired location with guide wires or other means known in the art. Introducer sheath 32 is conventional in the art and may include a handle 34 or hub at a proximal end of a sheath 32. The handle 34 may contain a hemostasis valve and movement of sheath 32 may be controlled through handle 34. Handle 34 also defines a lumen coaxially aligned with sheath and through which shaft 26 extends and additional lumens for passage of conductors and fluids to or from electrical and fluid connectors.

Drive housing 14 is provided to house mechanical and possibly electrical components used to generate a motive force in driving member 16. Housing 14 provides structural support for the components and protects the components from foreign objects and elements. Housing 14 also provides a base for mounting components of drive interface kit 20 as described in greater detail hereinbelow. Drive housing 14 may be made from conventional plastics. Referring to FIG. 2, in the illustrated embodiment housing 14 includes a base 36 and a lid 38. Base 36 defines a bottom wall 40 and a plurality of side walls 42, 44, 46, 48 extending generally perpendicular from bottom wall 40. Base 36 further defines a connector 50 extending from side wall 42 for a purpose described hereinbelow. Lid 38 defines a top wall 52 and a plurality of side walls 54, 56, 58, 60 configured to engage side walls 42, 44, 46, 48 of base 36. Top wall 52 includes an opening through which driving member 16 extends. Lid 38 may also define a pair of notches 62, 64 on opposite sides of lid 38. Notches 62, 64 each define an inclined surface 66 angling from top wall 52 inward towards the center of lid 38 for a purpose describe hereinbelow. When coupled together, base 36 and lid 38 define an enclosed space in which one or more components used to impart motive force to driving member 18 are located. These components may include, for example, one or more shafts and gears. The components may also include a conventional motor. Alternatively, the motor could be located external to drive housing 14 with a motor shaft extending into housing 14. Drive housing 14 may include an electrical connector through which control signals (including, for example, motor control signals) and feedback signals (including, for example, motor position sensor signals) may be exchanged with controller 18.

Driving member 16 is provided to generate a motive force. In the illustrated embodiment, driving member 16 comprises a cogwheel configured for rotation about a rotational axis 68 and with a set of radially outwardly extending teeth 69. It should be understood, however, that driving member 16 could assume a variety of forms. Driving member 16 may be driven by a motor within or external to drive housing 14. Driving member 16 could alternatively be driven by a motive force provided by manually by a clinician.

Controller 18 provides a means for controlling the operation of various components of system 10 and device 12. Controller 18 may also perform a variety of tasks depending on the nature of device 12 such as determining the geometry or EP characteristics of a region of interest, generating images of the region of interest for output to a display (not shown) or controlling the delivery of ablative energy to the region of interest. Controller 18 may comprise a programmable microprocessor or microcontroller or may comprise an application specific integrated circuit (ASIC). Controller 18 may include a central processing unit (CPU) and an input/output (I/O) interface through which controller 18 may receive a plurality of input signals including signals generated by device 12 and by motor position sensors (e.g. a rotary encoder) and generate a plurality of output signals including those used to control and/or provide data to the motor and device 12. Controller 18 may implement a dead man's switch and/or velocity limit switches as failsafe mechanisms for controlling device 12.

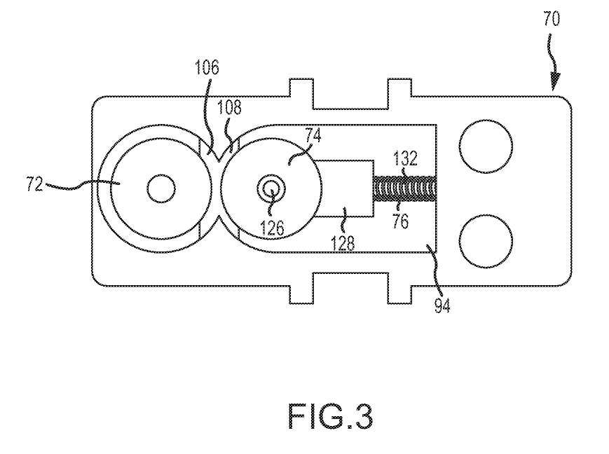

Referring again to FIG. 1, drive interface kit 20 is provided as a disposable interface between the other components of drive system 10 and device 12. Kit 20 includes a detachable housing 70, driven member 72, and idler member 74. Kit 20 may also include means, such as spring 76 (shown in FIG. 3) for biasing idler member 74 in a direction towards driven member 72, means, including actuator 78 and retainer 80, for urging idler member 74 in a direction away from driven member 72 against the force of spring 76, and coupling member 82.

Housing 70 provides structural support for idler member 74 and provides a guide for device 12. Housing 70 may be made from conventional plastics. In the illustrated embodiment, housing 70 includes a top wall 84 and a plurality of side walls 86, 88, 90, 92 generally perpendicular to top wall 84. Together, top wall 84 and side walls 86, 88, 90, 92 define an inner side 94 (see FIG. 3) and outer side 96 to housing 70.

Top wall 84 defines a channel 98 extending longitudinally from side wall 86 to side wall 88 and disposed between side walls 90, 92 that is configured to receive and guide medical device 12 and, particularly, shaft 26 of device 12 in the illustrated embodiment. Channel 98 is open at the top and defines a bottom 100 and opposed sides 102, 104. Sides 102, 104 of channel 98 include apertures 106, 108 through which driven and idler members 72, 74 may extend through to engage shaft 26 of device 12. Top wall 84 also includes a slot 110 extending perpendicular to channel 98 on the side of channel 98 on which idler member 74 is disposed and extending between the inner and outer sides 94, 96 of housing 70. Slot 110 is configured to receive an inwardly extending post (not shown) or similar structure connecting actuator 78 to retainer 80 for a purpose described hereinbelow.

Side walls 86, 88, 90, 92 are configured to engage top wall 52 of lid 38 of drive housing 14. Opposing side walls 86, 88 each include a pair of outwardly extending posts 112 configured to receive pins (not shown) extending therebetween on which clips 114, 116 are mounted for rotation about a longitudinal axis of the pins. Clips 114, 116 are generally S-shaped in cross-section and are configured such that a lip 118 on one end of each clip 114, 116 engages a corresponding inclined surface 66 of a corresponding notch 62, 64 upon rotation of clips 114, 116 in one rotational direction in order to attach housing 70 to drive housing 14. Rotation of clips 114, 116 in the opposite rotational direction disengages lip 118 from surface 66 to permit detachment of housing 70 from drive housing 14 for later disposal of housing 70. Rotation of clips 114, 116 may be affected by applying torque to a lip 120 at an opposite end of either clip 114, 116 relative to lip 118. Although FIGS. 1-3 illustrate a particular means for attaching housing 70 to housing 14 including rotatable clips 114, 116 mounted on housing 70 and configured to engage inclined surfaces 66 in housing 14, it should be understood that a variety of means for attaching housing 70 to housing 14 could be employed without departing from the spirit of the present invention including, for example, aligned posts and bores, temporary fasteners such as screws and other structures. Upon attachment of housing 70 to drive housing 14, one end of each side wall 86, 88, 90, 92 opposite top wall 84 engages top wall 52 of lid 38 of housing 14 such that inner side 94 of housing 70 is configured to define an enclosed space containing driving member 16 and partially containing driven and idler members 72, 74.

Driven member 72 provides a means for imparting a motive force to device 12, and particularly shaft 26 of device 12, to move device 12 longitudinally, thereby advancing or retracting shaft 26 within introducer sheath 32 and the body. Driven member 72 may be made from conventional plastics. Driven member 72 is configured for movement by driving member 16. In the illustrated embodiment, driven member 72 comprises a wheel and is configured for rotation by driving member 16. A radially inner surface of the wheel defines a hub from which a plurality of radially inwardly extending teeth 122 extend that are configured to mesh with teeth 69 of driving member 16. A radially outer surface of the wheel defines a groove 124 with a surface configured to engage medical device 12 and, in particular, shaft 26 of device 12. The side walls of groove 124 formed by opposed lips at either axial end of the wheel may be curved such that groove 124 is substantially concave or may be straight such that groove is substantially rectilinear in cross-section. Upon attachment of housing 70 to drive housing 14, driven member 72 is configured to be partially disposed within the enclosed space between inner side 94 of housing 70 and top wall 52 of lid 38 of housing 14. Driven member 72 is further configured to extend into channel 98 through aperture 106 in side 102 of channel 98 to engage device 12.

Idler member 74 provides a means for maintaining engagement of device 12 with driven member 72. Idler member 74 may be made from conventional plastics. Referring to FIG. 3, idler member 74 comprises a wheel in the illustrated embodiment and is supported within housing 70 on a post 126 that extends downwardly from a movable plate 128. A radially outer surface of idler member 74 defines a groove 130 with a surface—opposing the surface defined by groove 124 of driven member 72—configured to engage medical device 12 and, in particular shaft 26 of device 12. The side walls of groove 130 formed by opposed lips at either axial end of the wheel may be curved such that groove 130 is substantially concave or may be straight such that groove is substantially rectilinear in cross-section. Upon attachment of housing 70 to drive housing 14, idler member 74 is configured to be partially disposed within the enclosed space between inner side 94 of housing 70 and top wall 52 of lid 38 of housing 14. Idler member 74 is further configured to extend into channel 98 through aperture 108 in side 104 of channel 98 to engage device 12. Although driven member 72 and idler member 74 comprise wheels in the illustrated embodiment, it should be understood that driven member 72 and idler member 74 could take on other forms sufficient to provide opposed surfaces engaging device 12 including, for example, conveyor belts.

Spring 76 is provided to bias idler member 74 in a first direction towards driven member 72 such that opposed surfaces in driven and idler members 72, 74 securely engage device 12. Spring 76 may comprise a compression spring or a formed flat spring. Referring to FIG. 3, spring 76 may be disposed about a rod 132 connecting retaining member 80 and plate 126 with spring 76 bearing on opposed surfaces on inner side 94 of housing 70 and plate 126.

Actuator 78 and retainer 80 are provided to selectively urge idler member 74 in a direction away from driven member 72 against the force of spring 76 and may be made from conventional plastics. Retainer 80 is configured to receive one end of rod 132. Actuator 78 is coupled to retainer 80 by a post extending through slot 110 in top wall 84 of housing 70. Using actuator 78, a clinician can move retainer 80 in a direction away from idler member 74 to overcome the force of spring 76 and move rod 132, plate 128 and idler member 74 in the same direction to permit insertion or removal of medical device 12 from channel 98. It should be understood that, although actuator 78 and retainer 80 have a particular form illustrated in the drawings, a variety of structures could be used to provide an opposing force to spring 76.

Coupling member 82 provides a means for supporting introducer sheath 32 in a proper position and orientation relative to channel 98 in housing 70. Coupling member 82 may be made from conventional plastics and is configured for attachment to one of housings 14, 70. In the illustrated embodiment member 82 includes a connector 134 at one end configured to engage connector 50 extending from base 36 of drive housing 14 with the connector 134 in member 82 defining a rectilinear recess configured to receive connector 50 on drive housing 14. It should be understood that the size, shape and form of connectors 50, 134 could be varied without departing from the spirit of the invention. At an opposite end of member 82 from connector 134, side walls 136, 138, and a rib 140 extending between side walls 136, 138 define a cradle or recess 142 configured to receive one end of sheath 32 such as handle 34 of sheath 32. Recess 142 may be curved or otherwise configured for a particular sheath 32 and a center of recess 142 is aligned with channel 98 to prevent misalignment of device 12.

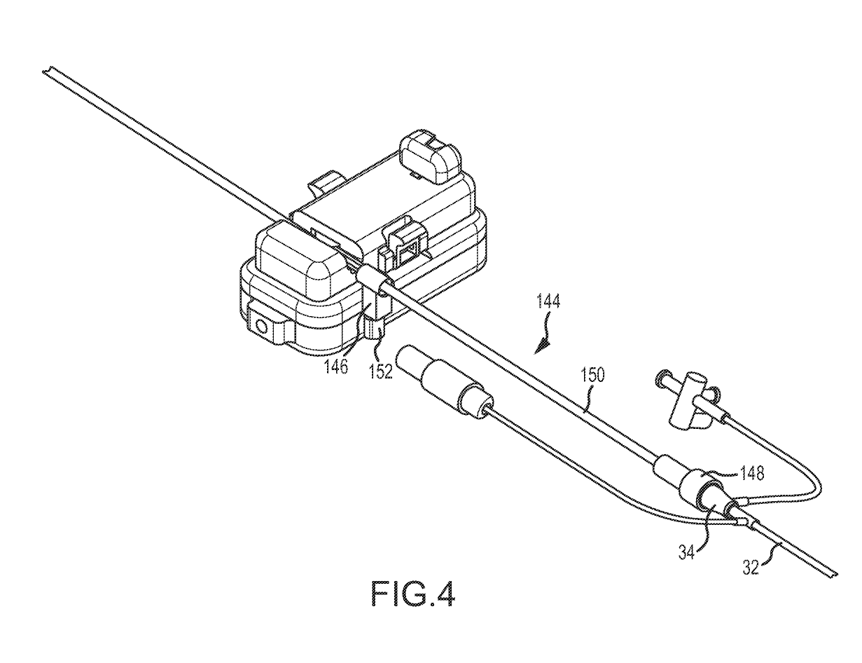

Referring to FIG. 4, in an alternative embodiment of the invention, a coupling member 144 includes a pair of connectors 146, 148 and an intermediate tubular member 150 disposed between connectors 146, 148 that is configured to receive device 12. Connectors 146, 148 and member 150 may be made from conventional plastics. Connector 146 is configured for attachment to one of housings 14, 70. In the illustrated embodiment, connector 146 is attached to drive housing 14 and supported on a flange 152 extending from side wall 42 of base 36 housing 14 through, for example, a pin and hole arrangement. Connector 146 defines a tubular passage at an one end configured to support one end of tubular member 150 and provide a junction at which device 12 enters tubular member 150. Connector 148 is disposed at an opposite end of tubular member 150 and defines a hub configured to receive one end of sheath 32 such as a handle 34 of sheath 32.

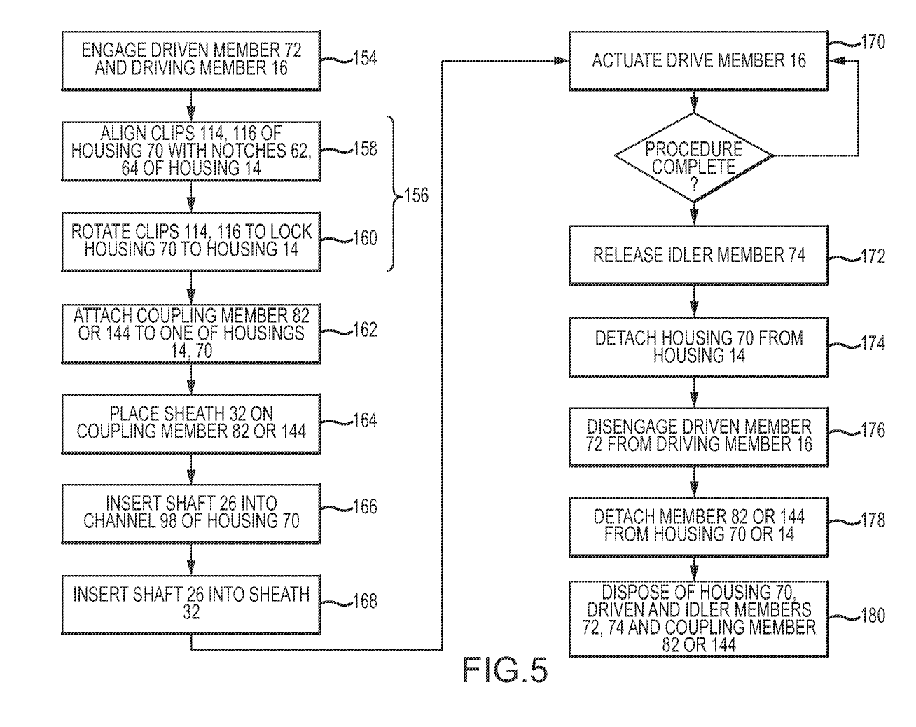

Referring to FIG. 5, a method for controlling longitudinal movement of an elongate medical device such as device 12 is described. The method may begin with the step 154 of engaging driven member 72 with driving member 16 for rotation therewith. The method may continue with the step 156 of attaching detachable housing 70 to drive housing 14. Step 156 may include the substeps 158, 160 of aligning notches 62, 64 and clips 114, 116 and then rotating clips 114, 116 to lock housing 70 in position relative to housing 14. Idler member 74 may be mounted on post 126 prior to attachment of housing 70 to housing 14 such that idler member is biased towards driven member 72 by spring 76 upon attachment of housing 70 to housing 14. The method may continue with the steps 162, 164 of attaching coupling member 82 or 144 to one of housing 70 and housing 14 (if coupling member 82 or 144 is not already attached to housing member 70) and placing a tubular body such as sheath 32 through which device 12 may be moved longitudinally on coupling member 82 or 144. In particular, handle 34 may be inserted into recess 142 of coupling member 82 or into hub 148 of coupling member 144. The method may continue with the step 166 of inserting shaft 26 of device 12 into said channel 98 of housing 70 between opposed surfaces of driven member 72 and idler member 74 and the step 168 of inserting shaft 26 of device 12 into sheath 32 (and particularly handle 34 of sheath 32). The method may then continue with the step 170 of actuating drive member 16 to move device 12 in a longitudinal direction. As discussed hereinabove, movement may be caused by motor or another motive power source (including, for example, by hand). Shaft 26 may be advanced and retracted in at various times during the procedure. Upon completion of the procedure, the method may continue with the step 172 of urging idler member 74 in a direction away from driven member 72. As discussed hereinabove, actuator 78 may be moved to compress spring 76 and move idler member 74 in a direction away from driven member 72. The method may then continue with the step 174 of removing device 12 from channel 98 and from sheath 32 (as well as removing sheath 32 from coupling member 82 or 144). The method may then conclude with the steps 176 and 178 of detaching housing 70 from drive housing 14 (e.g., by rotating clips 114, 116 to disengage the clips 114, 116 from notches 62, 64) and disengaging driven member 72 from driving member 16. If coupling member 82 or 114 is attached to drive housing 14 as opposed to housing 70, an additional step 180 of detaching member 82 or 114 from housing 14 may also be required. Housing 70, driven member 72 (as well as idler member 74 and its mounting components) can then be disposed of while drive housing 14 may be reused. It should be understood that the above-described steps could occur in various orders and that the particular order illustrated in FIG. 5 is exemplary only. For example, the order of steps 156, 162 could be reversed as could the steps 176, 180. Steps 166 and 168 could also be reversed in order.

A disposable drive interface kit in accordance with the present invention is advantageous because it contains relatively few components and allows reuse of drive system components. The detachable housing locates the channel for device 12 in a separate, detachable, and disposable housing. As a result, the drive housing 14 does not come into contact with device 12 and does not require sterilization or disposal. Rather, the few components comprising kit 20 can be easily removed and disposed of to permit reuse of the drive system components.

Although several embodiments of this invention have been described above with a certain degree of particularity, those skilled in the art could make numerous alterations to the disclosed embodiments without departing from the scope of this invention. All directional references (e.g., upper, lower, upward, downward, left, right, leftward, rightward, top, bottom, above, below, vertical, horizontal, clockwise and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not as limiting. Changes in detail or structure may be made without departing from the invention as defined in the appended claims.

What is claimed is:

1. A disposable drive interface kit for controlling longitudinal movement of an elongate medical device, said kit configured for attachment to a drive housing from which a driving member projects, said kit comprising:
a detachable housing configured for removable attachment to said drive housing, said detachable housing having an inner side and an outer side, said inner side configured to face said drive housing upon attachment of said detachable housing to said drive housing and said outer side defining a channel having a bottom and first and second opposed sides extending from said bottom and configured to receive said elongate medical device;
a driven member configured for movement by said driving member and extending into said channel on said first side of said channel, said driven member defining a first surface configured to engage said elongate medical device; and,
an idler member extending into said channel on said second side of said channel, said idler member defining a second surface opposing said first surface and configured to engage said elongate medical device.

2. The drive interface kit of claim 1, further comprising a coupling member configured for attachment to one of said detachable housing and said drive housing and configured to support a tubular body through which said elongate medical device may be moved longitudinally.

3. The drive interface kit of claim 2 wherein said tubular body comprises a sheath.

4. The drive interface kit of claim 2 wherein said coupling member comprises:
a first connector configured for attachment to said one of said detachable housing and said drive housing;
a second connector configured to receive said tubular body; and,
an intermediate tubular member disposed between said first and second connectors and configured to receive said elongate medical device.

5. The drive interface kit of claim 2 wherein said coupling member defines a recess configured to receive one end of said tubular body.

6. The drive interface kit of claim 5 wherein said recess is aligned with said channel.

7. The drive interface kit of claim 1, further comprising means for biasing said idler member in a first direction towards said driven member.

8. The drive interface kit of claim 7, further comprising means for urging said idler member in a second direction away from said driven member against a force of said biasing means.

9. The drive interface kit of claim 1 wherein said driven member and said idler member comprise wheels.

10. The drive interface kit of claim 1 wherein said inner side defines an enclosed space containing said driven member upon attachment of said detachable housing to said drive housing and said driven member and said idler member are partially disposed within said enclosed space.

11. The drive interface kit of claim 1 wherein said driven member extends through an aperture in said first side of said channel.

12. The drive interface kit of claim 11 wherein said idler member extends through an aperture in said second side of said channel.

13. A method of controlling longitudinal movement of an elongate medical device, comprising the steps of:
attaching a detachable housing to a drive housing from which a driving member projects, said detachable housing having an inner side and an outer side, said inner side configured to face said drive housing upon attachment of said detachable housing to said drive housing and said outer side defining a channel having a bottom and first and second opposed sides extending from said bottom and configured to receive said elongate medical device,
engaging a driven member with said driving member for rotation therewith, said driven member extending into said channel on said first side of said channel and opposing an idler member extending into said channel on said second side of said channel;
inserting said elongate medical device into said channel between opposed surfaces of said driven member and said idler member
actuating said drive member to move said elongate medical device in a longitudinal direction;
removing said elongate medical device from said channel; and,
detaching said detachable housing from said drive housing.

14. The method of claim 13, further comprising the step of disengaging said driven member from said driving member.

15. The method of claim 13, further comprising the step of placing a tubular body through which said elongate device may be moved longitudinally on a coupling member attached to one of said detachable housing and said drive housing.

16. The method of claim 15 wherein said tubular body comprises a sheath.

17. The method of claim 15, wherein said idler member is biased in a direction towards said driven member upon attachment of said detachable housing to said drive housing.

18. The method of claim 15, further comprising the step of urging said idler member in a direction away from said driven member.

19. The method of claim 13 wherein said driven member and said idler member comprise wheels.

20. The method of claim 13 wherein said inner side defines an enclosed space containing said driven member upon attachment of said detachable housing to said drive housing and said driven member and said idler member are partially disposed within said enclosed space.
